(19) United States
(12) Patent Application Publication
(10) Pub. No.: US 2002/0106745 A1
(43) Pub. Date: Aug. 8, 2002

(54) Predicting the outcome of virus infections

(76) Inventors: Adrian V.S. Hill, Oxford (GB); Mark R. Thursz, London (GB); Howard C. Thomas, London (GB); Lyna Zhang, Oxford (GB); Angela Frodsham, Oxford (GB); Steven Best, Oxford (GB)

Correspondence Address: NIXON & VANDERHYE P.C., 8th Floor, 1100 North Glebe Rd., Arlington, VA, 22201-4714, US

(21) Appl. No.: 09/777,924

(22) Filed: Feb. 7, 2001

Related U.S. Application Data
(63) Continuation of application No. PCT/GB99/02603, filed on Aug. 9, 1999.

(30) Foreign Application Priority Data
Aug. 7, 1998 (GB) 9817266.1

(51) Int. Cl. C12Q 1/70; C12Q 1/68; C07H 21/04; C12P 19/34; A61K 39/12; A61K 39/29; C07K 16/00

(52) U.S. Cl. 435/91.1; 435/5; 435/6; 536/23.72; 424/227.1; 435/91.33; 424/204.1; 530/389.1

(57) ABSTRACT

The present invention provides methods of predicting those individuals likely to develop persistent infection after exposure to a virus such as the hepatitis virus, particularly the hepatitis B virus. In one embodiment, the method comprises determining whether the subject carries one or more alleles associated with altered clearance of the virus. In another embodiment, the method comprises determining whether the subject carries one or more alleles associated with altered secretion of a cytokine.

The present invention relates to methods of predicting those individuals likely to develop persistent infection after exposure to the hepatitis virus, particularly the hepatitis B virus,

The cytokine IL10, (also known as cytokine synthesis inhibitory factor) is produced by T_{H2} cells, a subset of T cells which favour antibody production (Roitt, Bostoff & Male-fifth Edition, Mosby). ILIO inhibits the production of the IFN-gamma, by inhibiting the development of interferon secreting lymphocytes (TH1 lymphocytes). It also inhibits the production of the cytokines IL-1, IL-6 and TNF-alpha by macrophages, and favours antibody type immune responses during infection.

Chronic infection by one of the Hepatitis viruses leads to liver cirrhosis and hepatocellular carcinoma in a significant proportion of cases. THI lymphocytes are thought to be essential for the control of viral replication and the elimination of hepatocytes infected with the hepatitis B virus (Penna et al., Hepatology, 25(4):1022-7 (1997)). To date patients infected with the virus may be treated with either interferon alpha or lyphoblastoid interferon. However, the response rate for this therapy is limited, e.g only around 40% in the case of chronic HBV Additionally, this treatment is expensive and thus there are pressures to rationalise the use of such treatment within the healthcare industry.

There is a point mutation at position 1082 (with respect to the transcriptional start site), (IL10 1082G*) which appears to be of functional significance: An adenine to guanine substitution is associated with increased levels of IL10 secretion (Turner et al., Eur. J. Immunogenet., 24(l):1-8 (1997)).

We have shown that the IL101082A* allele (low IL10 secretion level) is associated with persistent infection of hepatitis B virus in two totally independent populations of individuals. Thus we conclude that the IL101082 guanine allele (IL10 1082 G*) is associated with the clearance of HBV. This affects the prognosis or treatment of an individual patient subject to HBV infection.

Polymorphism of any cytokine or cytokine promoter including IL2, IL4, ILS, IL6, IL10, IL12, and also alpha interferon subtypes, gamma interferon could also be expected to influence the outcome not only of hepatitis B infection, but also hepatitis C, hepatitis G, human papilloma virus, human immunodefiency virus and other persistent virus infections.

Thus, in a first aspect the present invention provides, a method for predicting the outcome of a virus infection in a subject, comprising the step of determining whether the subject carries one or more alleles associated with altered clearance of said virus.

In this context, predicting the outcome of a virus infection means predicting the susceptability of a subject to infection by a virus (following exposure) and/or predicting the susceptability of a subject to suffer disease/damage as a result of infection.

The term ‘altered clearance’ in the present context means that the allelic variation is associated with an alteration in the natural or normal clearance rate of the virus. This may occur as a result of an altered secretion of the cytokine, for instance.

In a second aspect, the present invention provides a method for predicting the outcome of a virus infection in a subject, comprising the step of determining whether the subject carries one or more alleles associated with altered secretion of a cytokine.

In one embodiment of these aspects of the invention, the virus infection is a hepatitis virus infection, particularly hepatitis B. In the case of the latter the method comprises determining whether the subject carries the IL10 A* allele, or the IL10G* allele.

As described above, the presence of either allele effects an individual's susceptibility or resistance to infection/disease.

The preferred method of carrying out the determination is to analyse a sample of the subject's DNA. Such a sample can conveniently be obtained from a biological sample, e.g. blood or a tissue sample.

The subject is preferably a human.

Suitably, the DNA obtained from the biological sample will be amplified using techniques well known to those skilled in the art, e.g. PCR techniques (Sambrook et al., Molecular Cloning, third edition—Cold Spring Harbor Labs Press,). For example the IL10 gene region and more particularly the IL10 promoter region, can be amplified. Such techniques will involve the use of at least one pair of suitable primers. Suitable primers can be chosen on the basis of the DNA sequence coding for the cytokine in question. In the case of the ILIO gene, suitable primers include the following:

| | |
|---|---|
| 5′ CTG GCT CCC CTT ACC TTC TAC ACA 3′ | SEQ ID NO.1 |
| 5′ TGG GCT AAA TAT CCT CAA AGT TCC 3′. | SEQ ID NO.2 |

These primers are designed to amplify a 656bp sequence of the DNA that includes the IL10 1082 point mutation.

Suitably, the presence of the point mutations will be detected using a sequence specific oligonucleolide hybridisation technique, as described herein. Such a technique will involve the use of suitable probes which will be chosen on the basis of the DNA sequence coding for the cytokine in question. In the case of the IL10 gene 1082 polymorphism, suitable probes include the following:

| | |
|---|---|
| 5′ TTT GGG AGG GGG AAG 3′ | SEQ ID No.3 |
| 5′ TTT GGG AAG GGG AAG 3′ | SEQ ID No.4 |

In the context of the present invention, IL10 gene region can mean the whole of the IL10 gene, or, alternatively, a part thereof. Clearly, however, if only a part is amplified it should include that portion of the gene associated with a particular point mutation, polymorphism etc. For instance in the case of the IL10 1082A*/IL1082G* allele, the portion of the gene which is amplified must include the promoter and may also include the coding region.

In further aspects, the present invention provides nucleic acid sequences comprising at least one of the sequences as set out in SEQ ID No. 1, SEQ ID No. 2, SEQ ID a No. 3 or SEQ ID No. 4, or a fragment thereof comprising at least nine nucleotides. The use of nucleic acid sequences in predicting the outcome of a virus infection by determining whether a subject carries one or more alleles associated with altered clearance of said virus. The use of nucleic acids in predicting the outcome of a virus infection by determining whether the subject carries one or more alleles associated with altered secretion of a cytokine.

Preferably, the nucleic acid sequence is one which hybridises to a flanking region of an allele associated with virus infection. Preferably, the allele is associated with infection by hepatitis, in particular hepatits B.

In a further aspect, the present invention provides a kit for use in a method for predicting the outcome of a virus infection in a subject which comprises one or more reagents for use in determining the presence or absence of one or more alleles associated with altered clearance of the virus.

In the context of the present invention a reagent includes one or more primers.

In yet a further aspect, the present invention provides a kit for use in a method for predicting the outcome of a virus infection in a subject which comprises at least one pair of primers suitable for PCR amplification of at least a portion of the gene coding for a cytokine, and/or at least one pair of probes suitable for oligonucleotide hybridisation to the cytokine DNA sequence.

In the context of the present invention, hybridisation means that one oligonucleotide sequence will specifically anneal to a complementary oligonucleotide sequence and will remain annealed under stringent conditions, for example, at 35 to 65° C. in a salt solution of about 0.9M.

Examples of suitable primers and probes are described herein.

Preferred features of each aspect of the invention are as for each other aspect, mutatis, mutandis.

The invention will now be described by reference to the following example, which should not be construed as in any way limiting the invention.

### EXAMPLE 1

Three point mutations in the IL10 promoter region have been described. These are at positions -1082G (G/A), -819 (CIT) and -592 (C/A) with respect to the transcription initiation site. Only the -1082 polymorphism has been shown to be of functional signficance.

PCR primers and conditions

The primers are designed to amplify a 656 bp sequence of DNA that includes all three of the point mutations. This fragment of the promoter region of human IL-10 gene, spanning -1179 to -523, was amplified by PCR with the use of 5′CTGGCTCCCCTTACCTTCTACACA3′ as a forward primer and 5′TGGGCTAAATATCCTCAAAGTTCC3′ as a reverse primer.

The reaction mix contains: 5 μl 10× PCR buffer (100 mM Tris-CHI, pH8.3, 500 mM KCl) 6 μl 25 MgCl₂ (3.0 M) 3 μ5mM dNTP nix (300 μM) 0.5 μl each primer (120 nM) 2 μl genomic DNA (5 ng) 34 μl H₂O 1 unit Taq Gold

PCR programne: 95° C. for 14 minutes for one cycle 95° C. for 15 s, 58° C. for 30 s, 72° C. for 30 s, for 35 cycles. 72° C. for 2 minutes for one cycle.

Allele identification

A sequence specific oligonucleotide hybridisation technique is used to identify the genotype.

Dot-blotting method

1. Add 10 μl of each PCR product to 76 μl TE buffer (Tris/EDTA pH 8), 6 μl 0.5M EDTA, and 8 μl of 6M NaOH.

2. Keep on ice for 10 minutes.

3. Add 100 μl 2M Ammonium Acetate and keep on ice till required.

4. Cut Nylon membrane to size and assemble dot-blot manifold.

5. Add 100 μl 2M Ammonium Acetate to each well of dot-blot apparatus followed by the PCR product mix (200 μl) and then a further 200 μl of 2M Ammonium Acetate.

6. Bake membrane for 2 hours at 80° C.

Hybridisation and washing

1. Block membrane with 10 mls of blocking solution for 30 minutes at room temp.

2. Prehybridise with 10 mls TMAC hybridisation solution for 45 minutes at 41° C. for -1082G and 43° C. for -1082A.

3. Hybridise at same temperatures with 10 mls TMAC bybridisation solution containing appropriate digoxigenin labelled probe (see below for sequence).

4. Wash with 25 ml wash buffer at room temp for 20 minutes.

5. Stringency wash with TMAC hybridisation solution for 15 minutes at 47° C. for -1082G and 48° C. for -1082A.

Detection

1. Rinse in buffer 1.

2. Block with 10 mls buffer 2 for 30 minutes at room temp.

3. Add 1 μl anti-dig-AP (Boehringer Mannheim) 30 minutes at room temp.

4. Wash with washing buffer 30 minutes at room temp.

5. Equilibrate with 10 ml buffer 3.

6. Add 5 ml of 1/100 dilution of CSPD solution (Boehringer Mannheim) for 2-10 minutes.

7. Wrap membrane in clingfilm and leave 15 minutes at 37° C.

8. Expose to Xray film for 10-15 minutes and develop film.

Stripping

1. Wash with 10 ml stripping buffer 1 for 30 minutes at 80° C.

2. Wash with 20 ml stripping buffer 2 for 10 minutes at room temp.

3. Wash with 20 ml stripping buffer 3 for 30 minutes at 37° C.

4. Rinse with SSC.

The procedure can now be repeated, using the same filter, with the second digoxigenin labelled probe, and films for the two alleles compared and the genotype recorded.

Probe Sequences and Solutions

| | |
|---|---|
| −1082G | TTT GGG AGG GGG AAG |
| −1082A | TTT GGG AAG GGG AAG |

Blocking solution

200 ml 20× SSPE 10 ml Blocking reagent stock (Boehringer Marnheim) 10 ml 10% laurylsarcosine Water to make 1000 ml.

TMAC hybridisation solution

600 ml 5M TMAC 50dm 1M Tris pH 8

10 ml 10% SDS 4 ml 0.5M EDTA Water to 1000 ml.

Wash buffer

100 ml 20×SSPE 10 ml 10% SDS Water to 1000 ml.

Buffer 2

50 ml 10× buffer 1 5 ml blocking reagent stock Water to 500 ml

10× buffer 1

^{87.65} g NaCl 116.1 g Maleic acid NaOH to pH 7.5 Water to 100 ml

20xSSPE

175.3 g NaCL, 175.4 31.2 g NaH₂PO₄2H₂O 7.4 g Na₂EDTA pH 7.4 with NaOH Water to 1000 ml

Washing buffer

100 ml 10× buffer 1

3 ml Tween 20 Water to 1000 ml

Buffer 3

100 ml 1M Tris pH 9.5 20 ml 5M NaCl 50 ml 1M MgCl₂ Water to 1000 ml

Stripping buffer 1

100 ml 0.5M EDTA pH 8 100 ml 20× SSC Water to 1000 ml

Stripping buffer 2

100 ml 20× SSC 10 ml 10% SDS Water to 1000 ml

Stripping buffer 3

33.4 ml 6M NaOH 10 ml 10% SDS Water to 1000 ml.

Results

European Subjects

IL10(-1082) in HBV

| Genotype | Acute N (%) | Chronic N (%) |
|---|---|---|
| AA | 13 (19.7) | 23 (36.5) |
| AG | 34 (51.5) | 29 (46.0) |
| GG | 19 (28.8) | 11 (17.5) |

Allele frequency analysis (A v G): p = 0.02

Gambian Subjects

Details of this Gambian case-control study have been described previously (Thursz M, New England Journal of Medicine, 332:1065-1069 (1995)). West African children age 1 to 10 years old, who attended to the hospitals and clinics for HBV unrelated conditions such as malaria, were recruited from hospital and clinics in the western, coastal region near the capital of the Gambia. Subjects were classified according to their serologic markers of HBV infection. The acute hepatitis patients who recovered from HBV infection were psotive for IgG HBV core antibody and negative for HBV surface antigen. The persistent carriers were positive for both HBV core antibody and surface antigen. Subjects with IgM HBV core antibodies and those with antibodies to HIV were not included in the study. Serological tests were carried out using standard ELISA kits (Boehringer Mannheim) (Hill, 1991). Statistical analysis was performed using a 2×2 chi-squared test to compare allele frequencies in the groups.

IL10(-1082) in HBV

| Genotype | Acute N (%) | Chronic N (%) |
|---|---|---|
| AA | 75 (38%) | 106 (48%) |
| AG | 84 (44%) | 94 (43%) |
| GG | 36 (18%) | 19 (9%) |

Allele frequency analysis (A v G): P = 0.003

The IL10 1082A* allele (low secretion level) was associated with persistent infection in two totally independent populations. We therefore conclude that IL10 1082G* is associated with clearance of HBV.

Having described the invention with particular reference to certain embodiments, it will be obvious to those skilled in the art to which the invention pertains after understanding the invention, that various changes and modifications may be made without departing from the spirit and scope of the invention as defined by the appended claims.

1. A method for predicting the outcome of a virus infection in a subject, comprising the step of determining whether the subject carries one or more alleles associated with altered clearance of said virus.

2. A method for predicting the outcome of a virus infection in a subject, comprising the step of determining whether the subject carries one or more alleles associated with altered secretion of a cytokine.

3. A method as claimed in claim 1 or claim 2 wherein the viral infection is a hepatitis viral infection, human papilloma virus infection or human immunodeficiency virus infection.

4. A method as claimed in claim 3 wherein the virus infection is a hepatitis viral infection.

5. A method as claimed in claim 4 wherein the hepatitis virus infection is a chronic hepatitis B viral infection.

6. A method as claimed in any one of claims 1 to 5 wherein the cytokine is, IL2, IL4, IL5, IL6, IL10, IL12, alpha interferon, including subtypes thereof, or gamma interferon.

7. A method as claimed in claim 6 wherein the cytokine is IL10.

8. A method as claimed in claim 7 wherein it is determined whether the subject carries the IL10 1082 A* allele or the IL10 1082 G* allele.

9. A method as claimed in any one of claims 1 to 8 wherein the determination is carried out using a biological sample.

10. A method as claimed in claim 9 wherein the biological sample is blood or a tissue sample.

11. A method as claimed in claim 10 wherein the biological fluid is blood.

12. A method as claimed in any one of claims 1 to 11 wherein the determination is carried out using DNA obtained from a biological sample.

13. A method as claimed in claim 12 the wherein the DNA is amplified using a pair of suitable primers.

14. A method as claimed in claim 13 wherein IL10 cytokine DNA is amplified using a pair of suitable primers.

15. A method as claimed in claim 14 wherein the pair of suitable primers comprise the sequences described by SEQ ID No. 1 and SEQ ID No.2

16. A method as claimed in claim 15 wherein the IL10 1082 A*, or IL10 1082 G* allele is detected using probes comprising the sequences described by SEQ ID NO. 3 and SEQ ID NO. 4.

17. Nucleic acid sequences comprising at least one of the sequences as set out in SEQ ID No. 1, SEQ ID No. 2, SEQ ID No. 4 or SEQ ID No. 5, or a fragment thereof comprising at least nine nucleotides.

18. The use of nucleic acid sequences in predicting the outcome of a virus infection by determining whether a subject carries one or more alleles associated with altered clearance of said virus.

19. The use of nucleic acid sequences in predicting the outcome of a virus infection by determining whether a subject carries one or more alleles associated with altered secretion of a cytokine.

20. The use, as claimed in claim 18 or claim 19, wherein the nucleic acid sequence is one which hybridises to a flanking region of an allele associated with virus infection.

21. The use, as claimed in any one of claims 18 to 20, wherein the allele is associated with infection by hepatitis, in particular hepatitis B.

22. The use, as claimed in any one of claims 18 to 21, wherein the nucleic acid is as claimed in claim 17.

23. A kit for use in predicting the outcome of a virus infection in a subject which comprises one or more reagents for use in determining the presence or absence of one or more alleles associated with altered clearance of the virus.

24. A kit for use in predicting the outcome of a virus infection in a subject which comprises at least one pair of primers suitable for PCR amplification of at least a portion of the gene coding for a cytokine, and/or at least one pair of probes suitable for oligonucleotide hybridisation to the cytokine DNA sequence.

25. A kit as claimed in claim 23 or claim 24 modified by any one or more of the features of any one or more of claims 2 to 8 and 12 to 22.
